(19) United States
(12) Patent Application Publication
(10) Pub. No.: US 2025/0137987 A1
(43) Pub. Date: May 1, 2025

(54) DEVICE AND METHOD FOR CONE PENETRATION TESTING OF GROUNDS

(71) Applicant: FRAUNHOFER-GESELLSCHAFT ZUR FÖRDERUNG DER ANGEWANDTEN FORSCHUNG E.V., München (DE)

(72) Inventors: Lennart SIEMANN, Bremerhaven (DE); Jan HEBIG, Bremerhaven (DE); Taisiya PEIN, Bremerhaven (DE); Aisgo WERPUP OGURO, Bremerhaven (DE); Benedict PREU, Bremerhaven (DE)

(73) Assignee: FRAUNHOFER-GESELLSCHAFT ZUR FÖRDERUNG DER ANGEWANDTEN FORSCHUNG E.V., München (DE)

(21) Appl. No.: 18/835,113

(22) PCT Filed: Feb. 3, 2023
(86) PCT No.: PCT/EP2023/052696

§ 371 Date: Aug. 1, 2024

(30) Foreign Application Priority Data
Feb. 3, 2022 (DE) 10 2022 201 173.2

(51) Int. Cl. G01N 33/24; G01V 1/16; G01V 1/20

(52) U.S. Cl. CPC G01N 33/24; G01V 1/166; G01V 1/20

(57) ABSTRACT

The invention relates to a cone penetration testing device comprising a probe, which has a probing cone (1), and in particular a skin friction sleeve (2), as well as an inclinometer and a pore pressure gauge, and comprising a transport linkage (4, 5, 6), which is configured to be connected to the probe at a first of the ends (7) thereof and to push the probe by applying pressure, into a soil region to be probed, as well as a seismic measuring device (9, 10, 11, 12) for capturing seismic signals. The object of capturing the seismic measurement data via a plurality of channels is achieved by the seismic measuring device comprising a plurality of seismic sensors (for example pressure sensors, accelerometers, geophones) (9, 10, 11, 12), which are integrated into one or more elements (4, 5, 6) of the transport linkage and arranged spaced apart from one another along the longitudinal direction of the transport linkage. The various seismic pressure sensors can feed the measurement data thereof into a shared signal conductor (15).

The invention resides in the field of measurement instrumentation and is used particularly advantageously during the probing of soils.

In the case of geotechnical investigations, for example of soils in the onshore and offshore areas, what is known as cone penetration testing, (CPT method) is frequently employed, in which a probe is pushed into the subsurface. The probe usually has a conical shape at the tip thereof, which has a 60° apex angle, and the measurement values for the push-in forces at the tip of the probe are measured when the probe is pushed in so as to obtain information about the properties of the soil.

The measured data is usually transmitted by means of a data cable to a measuring station, which is located on the Earth's surface, for example in a test vehicle or on a ship. If it is provided to use a transport linkage for introducing the probe into larger soil depths, which is successively assembled as the penetration depth progresses, such a data cable is run through the cavities of the tubular linkage or is already threaded through the transport linkage at the beginning of the probing process. In addition, the coiled rod method and the downhole CPT method exist, for example, as alternatives to the transport linkage.

If the probing apparatus is supplemented with a seismic pressure sensor or a geophone, which is likewise introduced into the soil, it is possible to consecutively carry out seismic investigations at various depths, with the probe remaining in situ in each case (seismic CPT). In the process, pulses are introduced into the soil from the Earth's surface or, in the case of offshore applications, from the ocean bed, and the signals arriving at the seismic sensor are measured.

A device and a method for carrying out geotechnical investigations are known from the publication DE 10 2020 001 184 A1, in which other methods are employed for transporting measurement data from a probing device to a data processing device. The document mentions the option, for example, of establishing a wireless data transport by means of a battery-operated transmitted that is integrated into a probe or, in another variant, to store measurement data in the probe and to read the data out later, after the probing process has been completed and the probe has been retrieved.

Against the background of the prior art, it is the object of the present invention to improve and expand, in particular, the measurement data capturing of seismic data and of the provision thereof to a data processing device in a cone penetration testing device.

The object is achieved according to the invention by a cone penetration testing device having the features of claim 1. The dependent claims represent advantageous embodiments of such a cone penetration testing device. The invention also relates to a method for cone penetration testing.

The invention thus relates to a cone penetration testing device comprising a probe, which has a probing cone, and in particular a skin friction sleeve, and furthermore in particular an inclinometer, and comprising a transport linkage, which is configured to be connected to the probe at a first of the ends thereof and to push the probe, while applying pressure, into a soil region to be probed, and a seismic measuring device for capturing seismic signals. According to the invention, the object is achieved by the seismic measuring device comprising multiple seismic sensors, which are integrated into multiple elements of the transport linkage and arranged spaced apart from one another along the longitudinal direction of the transport linkage.

The transport linkage can comprise a plurality of rods or tubes, which are arranged axially one behind the other and are connected to one another. These form respective elements of the transport linkage.

Due to the fact that multiple seismic sensors, for example geophones, multicomponent sensors, accelerometers are provided along the transport linkage and are simultaneously available for the measurement after having been introduced into the soil, seismic measurements can be carried out by way of sensors that are arranged at varying soil depths in a certain position of the transport linkage, for example during an interruption in the advancement of the probe. These measurements can be carried out simultaneously or in short succession for different, multiple or all seismic sensors. In this way, a plurality of measurement data can be obtained, which individually or collectively are meaningful for various soil depths. This significantly improves the measurement data acquisition of seismic data compared to known methods since the advancement of the probe or of the transport linkage does not have to be repeatedly interrupted in order to carry out the seismic measurements. In addition, the relative position of the seismic sensors with respect to one another along the transport linkage is known. Since seismic sensors are distributed among multiple elements of the transport linkage, sufficient distances between the sensors can be generated, which ensure a sufficiently high quality of the measurements. According to the method according to the invention, a single depth measurement suffices to obtain this information, the depth measurement allowing the depth of one of the seismic sensors beneath the surface to be measured, wherein the depths of the remaining sensors can be derived from the known relative positions. Using an inclinometer that is integrated into the probing device, the angle of the advancement direction can be monitored and measured so that more precise data can be ascertained in terms of the sensor depth.

Multiple seismic sensors can be distributed among multiple, in particular at least two or three elements of the transport linkage. The sensors can thus take on larger distances among one another, and it is already possible to carry out initial measurements before all elements of the transport linkage have been introduced into the soil.

A probing cone is arranged at the tip of the cone penetration testing device, which significantly facilitates the penetration of the probe and of the transport linkage into the soil. The probing cone is usually provided with a force measuring unit, which allows the push-in forces to be continuously measured. In addition, in a manner that is likewise known, a skin friction sleeve can be provided behind the probing cone, which allows the skin friction to be measured during the advancement movement of the probe. In addition, it is also possible to provide a measuring device for the pore pressure of water in the vicinity of the probing cone, typically between the probing cone and the skin friction sleeve, to be able to measure the pore pressure, as well as an inclinometer. The seismic sensors are suitable for recording various modes of seismic waves. It is possible for two or three, four or more than four such seismic sensors to be provided, which each capture both seismic P and S waves.

The cone penetration testing device can advantageously be configured in that at least some of the seismic sensors are arranged at the lateral surface of one or more elements of the transport linkage which are arranged one behind the other in the longitudinal direction. For this purpose, the elements of the transport linkage can be designed as tubes, which have boreholes penetrating the walls thereof, into each of which seismic sensors are inserted so as not to protrude beyond the tube wall in the radial direction.

The sensors can advantageously be arranged slightly behind the enveloping lateral surface of the elements of the transport linkage, and advantageously it may be provided that the surface of the seismic sensors, at the outer lateral surface of the element or elements of the transport linkage, is covered with a protective layer which is, in particular, made of a plastic material, and furthermore in particular of an epoxy resin. Such a coating can also be provided when the surface of the seismic sensors ends with the lateral surface of the elements of the transport linkage. The layer should then be designed so as to generate nor or only minimal additional frictional resistance during the advancement of the transport linkage.

The cone penetration testing device can furthermore advantageously be configured in that each of the seismic sensors is connected to a signal conductor for forwarding measurement signals to a second end of the transport linkage located opposite the probe-side first end or the probing tip and/or that each seismic sensor comprises a unit for digitizing and/or modulating signals for the transport via the signal conductor.

The measurement signals of the seismic sensors are to be conducted to the second end of the transport linkage located opposite the probing tip, at which a data processing device is made available. For this purpose, individual signal conductors can be provided for the individual seismic sensors; however, it is also possible to collectively utilize signal conductors of multiple seismic sensors for forwarding signals of multiple seismic sensors. For example, a line that includes multiple individual conductors, each of which conducts one of the signals of a seismic sensor, can be provided as the signal conductor. It is also possible for a conductor to be provided which supplies a supply voltage for the seismic sensors.

For this purpose, it may be provided, for example, that the signal conductor is integrated in each case into an element of the transport linkage and, in particular, is arranged in a centric cavity or in a wall of the element. The individual signal conductors can, for example, be threaded into the still unassembled transport linkage before the cone penetration testing is conducted and/or can have already been connected to the individual elements of the transport linkage. For this purpose, the signal conductors can, for example, be placed into an inner cavity of the tubular elements of the transport linkage or be attached to an inner wall of the elements or to the outside of the elements.

Particularly advantageously, it may be provided that the signal conductor is composed of signal conductor sections, which are each detachably connected to one another at the ends of the elements of the transport linkage, wherein the signal conductor sections end, in particular, in each case at plug connection elements, which are connected to a respective end of an element of the transport linkage. The plug connection elements can have a multipolar design so as to establish a connection for each seismic sensor and the signal conductor assigned thereto. Generally speaking, the signal conductor sections can, for example, be connected to one another by means of plug or screw connections or by way of bayonet catches. This eliminates the need to introduce a long signal conductor into the elements of the transport linkage which are used, before the transport linkage is introduced and the signal conductor is or signal conductors are each connected to the parts of the signal conductor/of the signal conductors which have already been introduced into the soil, and the individual elements of the transport linkage are added consecutively. As a result, it is not necessary, for example, to define the maximum measurement depth for the selection of the signal conductor in advance, but the overall length of the signal conductor can be adapted based on the development of the measuring project. In this way, the use of excessively long data cables is avoided, and the individual sections can be easily replaced in the event of a defect.

Instead of multiple parallel signal conductor sections, it is also possible to use a single signal conductor that is divided in the longitudinal direction, to which then one or more seismic sensors are connected. However, it is also possible for a dedicated signal conductor to be assigned to each of the seismic sensors. In this case, multiple signal conductors can also run parallel to one another through the transport linkage.

Advantageously, it may be provided that the signal conductor sections are formed by one or more electrically insulated electrical conductors and/or by light guides. Accordingly, the individual signal conductor sections can, for example, be assembled within the scope of plug connections, or they may also be soldered together, adhesively bonded in an electrically conducting manner or welded together. Screw connections are also advantageously conceivable. If the signal conductor is a light guide, connections that can be comfortably established for light guides are also known.

The seismic sensors can already be installed and connected to a signal conductor during the production of the elements of the transport linkage. If the signal conductor is multipolar, a seismic sensor can be identified by the conductor pole assigned thereto. In the case of a bus solution, an identification code can be assigned to each sensor for transmission.

The measurement signals can, for example, be transmitted in the form of voltage signals via the signal conductor or signal conductors, wherein the data, for example, can be transmitted by way of voltage levels, however also by way of AC voltage signals and, in encoded form, by way of frequencies or in digitized form. For this purpose, each of the seismic sensors, but also the probe or the probing cone and the skin friction sleeve, as well as the pore pressure measuring device, can comprise analog-to-digital converters for digitizing the measurement data. If the data of the individual seismic sensors is transmitted in encoded and digital form, or transmitted consecutively in a time division multiplexing method, it is also possible that only few signal conductors, and in the extreme case only a single signal conductor, are provided along the transport linkage for the data transport. Encoding the signals ensures that a respective sensor, and thus also a measurement location, can be assigned to the individual signals.

In a special embodiment of the invention, it may also be provided, for example, that the signal conductor sections are formed by the elements of the transport linkage. In this case, electrical signals, for example in the form of digital voltage signals, can be transmitted by the elements of the transport linkage.

In addition to a cone penetration testing device as described above, the invention also relates to a method for probing, using such a cone penetration testing device, wherein the signal conductor is composed of signal conductor sections, and wherein elements of the transport linkage are consecutively assembled as the penetration depth of the probe increases, and signal conductor sections are also connected to one another in the process.

The invention furthermore relates to a method for using a cone penetration testing device as described above, in which, after at least a part of the transport linkage has been introduced, the penetration movement of the probe is interrupted, and one or more seismic measurements are carried out, using multiple pressure sensors that are spaced apart from one another in the longitudinal direction of the transport linkage.

The invention will be shown and described hereafter based on exemplary embodiments in figures. In the drawings:

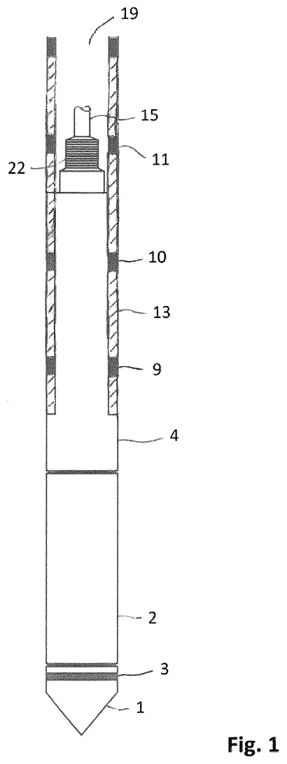

FIG. 1 shows a schematic longitudinal sectional view of a probe comprising an element of a transport linkage;

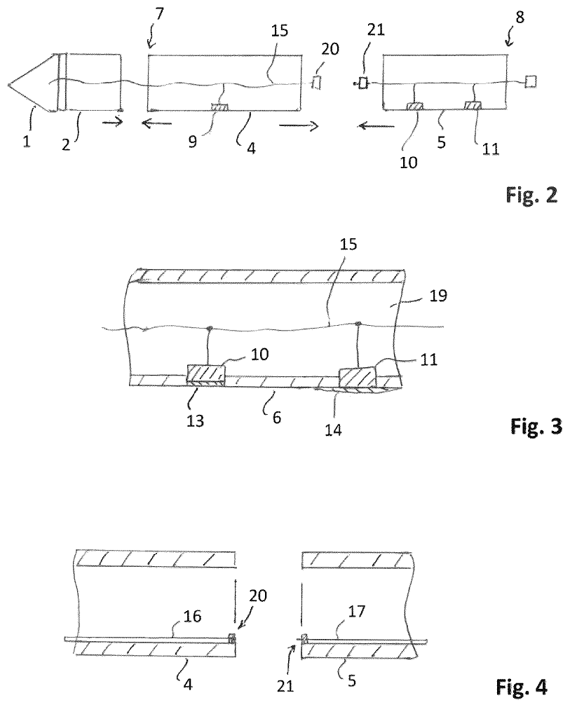

FIG. 2 schematically shows a probe comprising multiple elements of the transport linkage prior to assembly;

FIG. 3 shows a longitudinal sectional view of an element of the transport linkage comprising two seismic sensors;

FIG. 4 shows two elements of the transport linkage prior to being joined;

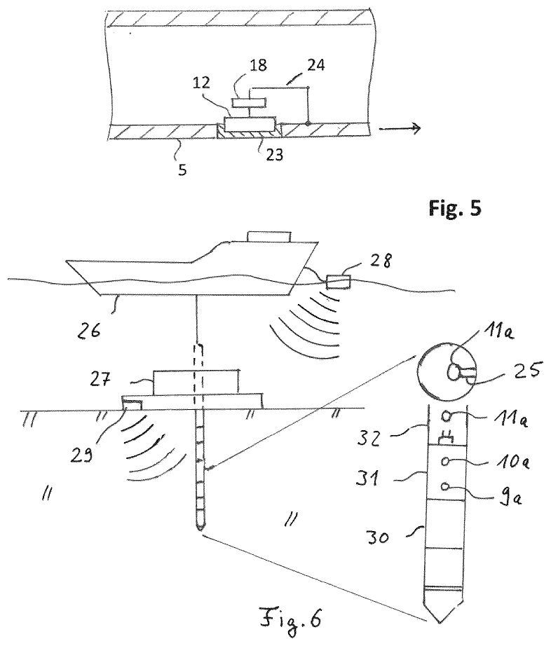

FIG. 5 shows an element of the transport linkage comprising a seismic sensor; and

FIG. 6 shows an offshore application as an application example.

FIG. 1 shows a partially longitudinally cut representation of a probe comprising a probing cone 1, a skin friction sleeve 2, and a filter element 3 for pore pressure measurement, which together form a probe that is pushed into soil to be investigated. The probe is connected to a first element 4 of a transport linkage designed as a tube. The seismic sensors/geophones 9, 10, 11 are arranged in the wall of the tube so as to be attached in openings penetrating the tube wall. Reference numeral 13 denotes an insulating layer, which covers and embeds the seismic sensors. A signal conductor 15 is arranged in the interior of the cavity 19 of the element 4 of the transport linkage. The signal conductor 15 is divided in the longitudinal direction corresponding to the lengths of the elements 4 of the transport linkage and, as will be described in greater detail below, the individual signal conductor sections are connected to one another by means of detachable connections. FIG. 1 shows a screw thread 22, which is used to join the signal conductor sections designed as coaxial conductors.

FIG. 2 schematically shows a probe 1, 2 and a transport linkage that is composed of two elements 4, 5 and has a first end 7 connected to the probe 1, 2 and a second end 8 opposite the probing tip, which is close to the Earth's surface or the soil surface during the measuring process, and in particular protrudes at the top from the soil. A signal conductor 15, which is joined by means of plug connection elements 20, 21 in each case at the transition between two elements 4, 5 of the transport linkage, extends in the longitudinal direction within the elements 4, 5 of the transport linkage. The signal conductor 15 can, for example, be designed as a single insulated electrical conductor, as a conductor bundle, or as a light guide. A design as a waveguide, for example in the form of a coaxial cable, is also conceivable.

Various seismic sensors 9, 10, 11 are connected to the signal conductor 15, which are distributed along the longitudinal direction of the transport linkage among various elements 4, 5 of the transport linkage. One or more seismic sensors 9, 10, 11 can be provided in a single element 4, 5.

The signal conductor can be formed by a signal bus, which can have a unipolar or a multipolar design and can contain a voltage supply unit used for the operation of communication modules of the individual seismic sensors, for example in the form of analog-to-digital converters, encoders, or frequency converters.

FIG. 3 shows two examples for the integration of seismic sensors into the wall of an element 6 of the transport linkage. A seismic sensor 10 is arranged in a continuous opening in the wall of the tubular element 6 in such a way that the surface thereof is recessed so as to be located behind the lateral surface of the tube. The resulting cavity is filled with a cover layer 13, for example made of epoxy resin, so that the sensor 10 is protected against environmental conditions. A second seismic sensor 11 is arranged in an opening of the element 6 so as to end with the circumferential surface of the element 6. In addition, a cover layer 14, for example made of epoxy resin, is provided which covers both the seismic sensor 11 and adjoining parts of the tubular element 6. The layer 14 should be dimensioned so as not to impair the penetration of the transport linkage into the soil, but thick and stable enough to protect the seismic pressure sensors. Each of the two sensors 10, 11 is connected to the signal conductor 15 in the cavity 19 of the element 6.

FIG. 4 shows an exemplary embodiment in which a signal conductor is made of two or more signal conductor sections 16, 17, which are fixedly connected to the wall of the elements 4, 5 of the transport linkage and, for example, are arranged in a groove on the inner side of the tube wall of the elements 4, 5 or attached to the inner side of the wall of the elements 4, 5 by adhesion. Each of the signal conductor sections 16, 17 ends in plug connection elements 20, 21, which are likewise fixedly connected to the elements 4, 5 of the transport linkage at the respective other ends so that the plug connection elements 20, 21 can likewise be reliably joined when the elements 4, 5 of the transport linkage are connected.

A signal conductor can also be held in the center of the cavity of the elements of the transport linkage, for example by way of spacers with respect to the cylindrical inner wall. In this way, damage to the signal conductor is prevented when two adjoining elements of the transport linkage are connected.

FIG. 5 shows one embodiment of the invention in which a seismic pressure sensor 12 is arranged in an electrically insulated manner in the wall of the element 5 of the transport linkage. An electrical insulation layer 23 is provided, which effectuates both electrical insulation of the sensor 12 with respect to the metallic tube of the element 5 and mechanical protection at the lateral surface of the element of the transport linkage.

The sensor 12 moreover comprises a converter 18, which converts the measurement signals into digital signals and which feeds the electrical signals thus formed into the element 5 of the transport linkage, or more precisely into the wall of the element 5, by means of a feed line 24. The elements 4, 5, 6 of the transport linkage can thus act overall as a signal conductor after having been joined. If, for example, the measurement signals are converted into a high-frequency digital signal, these signals can thus be transported without disruption via the transport linkage. It is thus also possible for the signals of various seismic sensors to be transmitted separately from one another by means of a time division multiplexing solution.

FIG. 6 shows an offshore application of a probe comprising multi-piece transport linkage, wherein a transmitter 28, 29 for seismic pulses can be provided on a ship 26 and/or on a foundation 27 on the floor of the ocean. The transport linkage 30 is illustrated in enlarged form in the right part of the figure. The transport linkage comprise a probing cone and elements 30, 31, 32 of the transport linkage, wherein one or more sensors 9a, 10a, 11a can be provided in each of multiple elements 31, 32 of the transport linkage.

In this exemplary embodiment, the sensors are arranged in the interior of the elements of the transport linkage and can be arranged and attached on the inside to the lateral surface of the elements, but also further radially to the inside, as is apparent from the cross-sectional representation for the sensor 11a, for example in the center on the central axis of the transport linkage, wherein the sensors can then be attached, for example, via holding elements, in particular webs or lugs 25 on the inner lateral surface of the elements of the transport linkage. The attachment can be configured in such a way that suitable acoustic coupling takes place.

1. A cone penetration testing device comprising a probe, which has a probing cone, and in particular a skin friction sleeve, and furthermore in particular an inclinometer, and comprising a transport linkage, which is configured to be connected to the probe at a first of the ends thereof and to push the probe, by applying pressure, into a soil region to be probed, and comprising a seismic measuring device for capturing seismic signals, characterized in that the seismic measuring device comprises a plurality of seismic sensors, which are integrated into a plurality of elements of the transport linkage and arranged spaced apart from one another along the longitudinal direction of the transport linkage.

2. A The cone penetration testing device according to claim 1, characterized in that at least some of the seismic sensors are arranged at the lateral surface of one or more elements of the transport linkage which are arranged one behind the other in the longitudinal direction.

3. The cone penetration testing device according to claim 2, characterized in that the surface of the seismic sensors, at the outer lateral surface of the element or elements of the transport linkage, is covered with a protective layer which is, in particular, made of a plastic material, and furthermore in particular of an epoxy resin.

4. The cone penetration testing device according to claim 1, characterized in that each of the seismic sensors is connected to a signal conductor for forwarding measurement signals to a second end of the transport linkage located opposite the probe-side first end and/or that each seismic sensor comprises a unit for digitizing and/or modulating signals for the transport via the signal conductor.

5. The cone penetration testing device according to claim 4, characterized in that the signal conductor is integrated in each case into an element of the transport linkage and, in particular, is arranged in a centric cavity or in a wall of the element.

6. The cone penetration testing device according to either claim 4, characterized in that the signal conductor is composed of signal conductor sections, which are each detachably connected to one another at the ends of the elements of the transport linkage, the signal conductor sections ending, in particular, in each case at plug connection elements, which are connected to a respective end of an element of the transport linkage.

7. The cone penetration testing device according to claim 4, characterized in that the signal conductor sections are formed by one or more electrically insulated electrical conductors and/or by light guides.

8. The cone penetration testing device according to claim 4, characterized in that the signal conductor sections are formed by the elements of the transport linkage.

9. A method for probing, using a cone penetration testing device according to claim 1, the signal conductor being composed of signal conductor sections, characterized in that elements of the transport linkage are consecutively joined as the penetration depth of the probe increases, and signal conductor sections are also connected to one another in the process.

10. A method for probing, using a cone penetration testing device according to claim 1, characterized in that, after at least a part of the transport linkage has been introduced, the movement of the probe is interrupted, and one or more seismic measurements are carried out, using a plurality of pressure sensors that are spaced apart from one another in the longitudinal direction of the transport linkage.
